(19) United States
(12) Patent Application Publication
(10) Pub. No.: US 2026/0174921 A1
(43) Pub. Date: Jun. 25, 2026

(54) VOLATILE COMPOSITION DISPENSER

(71) Applicant: The Procter & Gamble Company, Cincinnati, OH (US)

(72) Inventors: Pei LI, Oberursel (DE); Stephan Gary BUSH, Liberty Township, OH (US); Ashlesha TIPLE, Mason, OH (US); Ronald David TURNER, Cincinnati, OH (US); Faiz Feisal SHERMAN, Mason, OH (US)

(21) Appl. No.: 19/424,351

(22) Filed: Dec. 18, 2025

Related U.S. Application Data
(60) Provisional application No. 63/736,218, filed on Dec. 19, 2024.

(51) Int. Cl. A61L 9/03

(52) U.S. Cl. CPC A61L 9/037; A61L 9/032; A61L 2209/111; A61L 2209/132

(57) ABSTRACT

Energy efficient dispensers for delivering volatile compositions to the surrounding environment are provided. The dispensers employ a heatable evaporative member that has a large surface area in comparison to its footprint size. The dispenser can include a volatile liquid composition reservoir, a fluid transfer member in fluid communication with the volatile liquid composition reservoir; and a heating element affixed to the evaporative member. The evaporative member is in contact with the composition transfer surface, and heat is conductively transferred to at least the evaporative member.

### TECHNOLOGICAL FIELD

The present disclosure generally relates to dispensers for delivering volatile compositions into the environment in which the dispensers reside. The volatile compositions can provide a variety of different benefits including, for example, freshening or deodorizing.

### BACKGROUND

Plug-in air fresheners typically take the form of a wick-based dispenser with a heater that heats up a portion of the volatile liquid composition to the point it evaporates and travels into the surrounding area to provide a pleasing smell or other designed benefit. Many of the known dispensers have an inefficient system that loses heat to the environment without doing the useful work of heating the wick.

A need accordingly exists for more energy efficient dispensers that emit volatile compositions into the atmosphere.

### SUMMARY

Embodiments of the present invention are directed to energy efficient dispensers for delivering volatile compositions to the atmosphere and surfaces of objects located within a reasonable distance to the dispensers. The dispensers generally include a volatile liquid composition reservoir, a fluid transfer member in fluid communication with the volatile liquid composition reservoir and comprising a composition transfer surface, an evaporative member in contact with the composition transfer surface, and a heating element for transferring heat to the evaporative member. In one embodiment, the evaporative member comprises a mesh that is heatable by the heating element.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a perspective view of a first exemplary dispenser;

FIG. 2 is a perspective view of an exemplary dispenser evaporative member in the form of a woven mesh;

FIG. 3 is a perspective view of another exemplary dispenser evaporative member in the form of an expanded mesh;

FIG. 4 is a perspective view of an exemplary dispenser heating element;

FIG. 5 is a plan view showing an example of an integrally formed heating element and evaporative member;

FIG. 6 is a perspective view of a disk-shaped heating element affixed to a square-shaped mesh;

FIG. 7 is a side view of a second exemplary dispenser;

FIG. 8 is a side view of a third exemplary dispenser;

FIG. 9 is a side view of a fourth exemplary dispenser;

FIG. 10 is a side view showing a mesh evaporative member that comprises a base and two side panels;

FIG. 11 is perspective view of a dispenser for delivering two volatile compositions;

FIG. 12 is an exploded view of an exemplary dispenser comprising a housing component;

FIG. 13 is a graph showing perfume evaporation rates and profiles from an exemplary dispenser; and

FIG. 14 is a graph showing perfume evaporation rates and profiles from dispensers having two different power inputs.

### DETAILED DESCRIPTION

Reference within the specification to “form(s),” “aspect(s),” “embodiment(s)” or the like means that a particular material, feature, structure, and/or characteristic described in connection with the form/aspect/embodiment is included in at least one form/aspect/embodiment, optionally several forms/aspects/embodiments, but it does not mean that all forms/aspects/embodiments incorporate the material, feature, structure, and/or characteristic described. Furthermore, materials, features, structures, and/or characteristics may be combined in any suitable manner across different forms/aspects/embodiments, and materials, features, structures and/or characteristics may be omitted or substituted from what is described. Thus, forms/aspects/embodiments described herein may comprise or be combinable with elements or components of other forms/aspects/embodiments despite not being expressly exemplified in combination, unless otherwise stated or an incompatibility is stated.

Each of the terms “volatile composition(s)” and “volatile liquid composition(s),” as used herein, refers to a material that comprises a vaporizable material. The term “volatile composition(s),” thus includes, but is not limited to, compositions that are comprised entirely of a single volatile material. The terms “volatile materials,” “aroma,” “fragrance,” and “scents,” as used herein, include, but are not limited to pleasant or savory smells, and, thus, also encompass materials that function as insecticides, air fresheners, deodorants, aromacology, aromatherapy, insecticides, or any other material that acts to condition, modify, or otherwise charge the atmosphere or to modify the environment. It should be understood that certain volatile compositions including, but not limited to perfumes, aromatic materials, and scented materials, will often comprise one or more volatile materials (which may form a unique and/or discrete unit comprised of a collection of volatile materials). It should be understood that the term “volatile composition(s)” refers to compositions that have at least one volatile component, and it is not necessary for all of the component materials of the volatile composition to be volatile. The volatile compositions described herein may, thus, also have non-volatile components. It should also be understood that when the volatile compositions are described herein as being “emitted,” this refers to the volatilization of the volatile components thereof and does not require that the non-volatile components thereof be emitted. The volatile compositions of interest herein can be in any suitable starting form including, but not limited to, solids, liquids, gels, encapsulates, and combinations thereof, but may require transformation to a liquid form if not liquid at room temperature particularly when the dispenser utilizes a fluid transfer member, such as a wick, to transfer the volatile composition to an evaporative surface.

Many of the known dispensers have the heater positioned close to but not in contact with a wick containing the volatile liquid composition. In such an arrangement, the heater must first consume electrical energy to increase the temperature of the heater itself. Then, the heater increases the temperature of the air between the heater surfaces and the wick. Finally, enough heat is transferred to the wick so that its exposed surface releases a vapor as the volatile liquid composition evaporates. In this arrangement, heat is also lost to air which flows through the device due to buoyant effects and to adjacent dispenser components other than the wick. The useful work is to evaporate the volatile liquid composition, which requires only a fraction of the total energy supplied to the dispenser. Most of the energy is lost to the environment without doing useful work.

Energy efficient dispensers for delivering vaporized portions of a volatile composition into the air are provided. The dispensers incorporate innovative heat transfer design approaches that yield better performing dispensers, allowing for less energy consumption for a targeted benefit. It is contemplated that the volatile composition dispenser may be configured for use in a variety of applications to deliver the volatile composition to the air and/or ultimately to a surface.

The volatile composition dispenser may be configured as an electrical wall plug or batteryoperated dispenser. If one is used, the battery can be rechargeable or disposable. A computer device (e.g., computer, smart phone, tablet, etc.) may also power the dispenser in some forms.

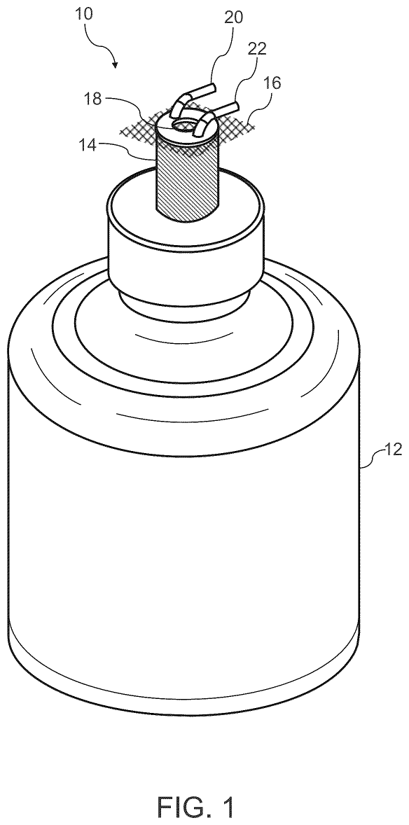

Dispensers provided herein generally comprise a reservoir for containing a volatile liquid composition, a fluid transfer member in fluid communication with the reservoir and including a composition transfer surface, an evaporative member in contact with the composition transfer surface, and a heating element for transferring heat to the evaporative member causing at least some of a contained volatile liquid composition to vaporize and be emitted from the dispenser. An exemplary dispenser 10 is shown in FIG. 1. Dispenser 10 includes a volatile liquid composition reservoir 12, a fluid transfer member 14 that is configured and positioned to access a liquid composition contained within reservoir 12, an evaporative member 16, and a heating element 18 comprising electrically connectable to a power source (not shown), such as through electrical leads 20 and 22. Fluid transfer member 14 includes a composition transfer surface. The evaporative member 16 may be placed onto or otherwise in contact with the composition transfer surface.

Reservoir 12 can comprise any suitable type of container and can be made of any suitable material. Suitable materials for the reservoirs include, but are not limited to, glass, plastic, compressed pulp (which may include a barrier coating layer), or mixtures thereof. The reservoir(s) can comprise any type of container that is suitable for holding volatile compositions. It is possible for a single reservoir to hold more than one type of volatile material. Such a reservoir could, for instance, have two or more compartments for volatile materials.

Fluid transfer member 14 may be configured in various ways. In one form, the fluid transfer member is a porous wicking element. Porous wicking elements can include different types of open cell porous media. These porous wicking media include, but are not limited to, open cell foams, felts, felts bounded with thermosetting resins, woven fibers, porous media comprising thermosetting resins and inorganic fillers, extruded plastic hollow tubes, porous media comprising synthetic and natural cellulosic materials, and mixtures thereof.

In some forms, a porous wicking element can comprise porous polymeric materials, including but not limited to, sintered porous polymeric materials. Plastics suitable for use in sintered polymeric materials of a porous wicking element can include polyolefins, polyamides, polyesters, rigid polyurethanes, polyacrylonitriles, polycarbonates, polyvinylchloride, polymethylmethacrylate, polyvinylidene fluoride, polyethersulfones, polystyrenes, polyether imides, polyetheretherketones, polysulfones, polyethersulfone, polyphenylene oxide, or combinations or copolymers thereof. In some aspects, a polyolefin comprises polyethylene, polypropylene, and/or copolymers thereof. The polyethylene can be high density polyethylene (HDPE), very high molecular weight polyethylene (VHMWPE), ultrahigh molecular weight polyethylene (UHMWPE), or mixtures thereof.

The fluid transfer member may comprise a fibrous material, including, for example, monocomponent fibers, bicomponent fibers, or combinations thereof. Monocomponent fibers can comprise polyethylene, polypropylene, polystyrene, nylon-6, nylon-6,6, nylon 12, copolyamides, polyethylene terephthalate (PET), polybutylene terephthalate (TBP), co-PET, or combinations thereof. The monocomponent fibers may be biodegradable, including those made from poly lactic acid (PLA), polyhydroxyalkanoates (PHA), polyhydroxybutyrate-valerate (PHBV), and polycaprolactone (PCL). Bicomponent fibers suitable for use in the fluid transfer member can comprise polypropylene/polyethylene terephthalate (PET); polyethylene/PET; polypropylene/Nylon-6; Nylon-6/PET; copolyester/PET; copolyester/Nylon-6; copolyester/Nylon-6,6; poly-4-methyl-1-pentene/PET; poly-4-methyl-1-pentene/Nylon-6; poly-4-methyl-1-pentene/Nylon-6,6; PET/polyethylene naphthalate (PEN); Nylon-6,6/poly-1,4-cyclohexanedimethyl (PCT); polypropylene/polybutylene terephthalate (PBT); Nylon-6/co-polyamide; polylactic acid/polystyrene; polyurethane/acetal; polylactic acid (PLA) copolymer/polylactic acid (PLA), and soluble copolyester/polyethylene.

The fluid transfer member can include pores or interstitial spaces (e.g., between adjacent fibers in a fiber bundle) of about 10 microns to about 200 microns, about 20 microns to about 150 microns, or about 30 microns to about 100 microns. A fluid transfer member can have an average pore volume from about 10% to about 70%, about 20% to about 60%, or about 30% to about 50%. Average pore size and average pore volume can be determined by a mercury porosimetry using the ASTM D4404 method.

As shown in FIG. 1, fluid transfer member 14 is in the form of a cylindrically-shaped wick. The wick may be defined by a length and a diameter or width, depending on the shape. The wick may have various lengths. For example, the length of the wick may be in the range of about 1 millimeter (“mm”) to about 100 mm, from about 5 mm to about 75 mm, or from about 10 mm to about 50 mm. The wick may have various diameters or widths. For example, the diameter or width of the wick may be at least 1 mm, or at least 2 mm, or at least 3 mm, or at least 4 mm. A wick may have a density in the range of about 0.100 grams/cm³ (“g/cc”) to about 1.0 g/cc.

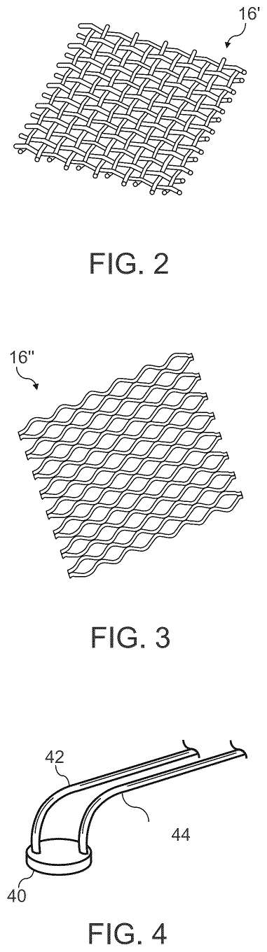

Evaporative member 16 is configured to receive liquid contained in volatile liquid composition reservoir 12 via fluid transfer member 14. With reference to FIG. 1, evaporative member 16 is in the form of a mesh. Such mesh designs suitable for the dispensers provided herein can have a variety of configurations, consist of one or multiple materials, and be manufactured with multiple processing techniques. A mesh can be a woven design, such as mesh 16′ illustrated in FIG. 2, or an alternative design, such as an expanded mesh 16″ shown in FIG. 3. The mesh examples in FIGS. 2 and 3 are provided merely to understand certain aspects of the inventive dispensers, and the scale of mesh components and the pores therebetween should not be overly interpreted or limited in any way to what is illustrated in the figures. By way of example and in contrast to what is shown in FIGS. 2 and 3, some useful meshes of the present disclosure are not see-through with the naked eye. And while the woven components in FIG. 2 appear to have a circular cross-section, they can also be elliptical, square, rectangular, triangular, or other geometrical shape.

When a mesh is used as the evaporative member, the mesh can be a “wickable mesh,” meaning a mesh that can have a wicking time of less than 30 minutes (1800 seconds (sec)), preferably less than 20 minutes (1200 sec), and more preferably less than 15 minutes (900 sec), as determined using the Method of Measuring the Wicking Time described below. A wickable mesh may have a wicking time of from about 1 to about 1800 sec, from about 1 to about 1200 sec, from about 1 to about 900 sec, from about 1 to about 700 sec, from about 1 to about 600 sec, from about 1 to about 550 sec, from about 1 to about 500 sec, from about 1 to about 400 sec, from about 1 to about 350 sec, from about 1 to about 300 sec, from about 1 to about 250 sec, from about 1 to about 200 sec, from about 25 to about 1800 sec, from about 25 to about 1200 sec, from about 25 to about 900 sec, from about 25 to about 700 sec, from about 25 to about 600 sec, from about 25 to about 550 sec, from about 25 to about 500 sec, from about 25 to about 400 sec, from about 25 to about 350 sec, from about 25 to about 300 sec, from about 25 to about 250 sec, from about 25 to about 200 sec, from about 50 to about 1800 sec, from about 50 to about 1200 sec, from about 50 to about 900 sec, from about 50 to about 700 sec, from about 50 to about 600 sec, from about 50 to about 550 sec, from about 50 to about 500 sec, from about 50 to about 400 sec, from about 50 to about 350 sec, from about 50 to about 300 sec, from about 50 to about 250 sec, from about 50 to about 200 sec, and any values within the foregoing ranges or any ranges created thereby.

The mesh can be made of any suitable material. It can be metallic or ceramic, for example. A mesh is preferably made from stainless steel.

In one form, the evaporative member comprises a woven mesh, preferably a woven wire mesh. Warps and wefts are the two basic components used in weaving to turn wires into a mesh. The lengthwise or longitudinal warps are held stationary in tension on a frame or loom while the transverse wefts (sometimes woof) are drawn through and inserted over and under the warp.

Mesh Count is the number of openings or apertures which can be counted per every linear inch of wire mesh. This is typically designated for both directions of the mesh. Therefore, a wire mesh which has ten openings per inch as measured across both its width and length would be designated a 10×10 mesh or Number 10 mesh.

An off-count mesh can be used in the dispensers provided herein. An “off-count mesh” is one which has a different number of openings per inch in one direction than another direction. Thus, a mesh with ten openings per inch measured in one direction and seventeen openings per inch measured in a second direction would be designated a 10×17 mesh.

Suitable meshes for use herein can have a number of openings per inch measured in a length direction of from 40 to 500, preferably from 50 to 400, and a number of openings per inch measured across a width direction of from 500 to 3000, preferably from 800 to 2500. The mesh may have a number of openings per inch measured in the length direction of from 40 to 500, from 40 to 400, from 40 to 300, from 40 to 200, from 40 to 100, from 40 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 300 to 500, from 300 to 400, from 400 to 500, and any values within the foregoing ranges or any ranges created thereby. The mesh may have a number of openings per inch measured in the width direction of from 500 to 3000, from 500 to 2500, from 500 to 2000, from 500 to 1500, from 500 to 1000, from 500 to 800, from 800 to 3000, from 800 to 2500, from 800 to 2000, from 800 to 1500, from 800 to 1000, from 1000 to 3000, from 1000 to 2500, from 1000 to 2000, from 1000 to 1500, from 1500 to 3000, from 1500 to 2500, from 1500 to 2000, from 2000 to 3000, from 2000 to 2500, from 2500 to 3000, and any values within the foregoing ranges or any ranges created thereby.

Woven mesh patterns suitable for the dispensers provided herein can include, but are not limited to, a plain Dutch weave, a reverse plain Dutch weave, a Dutch twilled weave, and a reverse Dutch twilled weave. Dutch twilled weaves are particularly useful in the dispensers disclosed herein because this pattern can provide a robust mesh in relation to its fineness. Dutch twilled weave meshes can have a fineness of 5 microns to 250 microns absolute opening, for example. Two exemplary meshes suitable for use in the dispensers include a 165×1,400 Dutch twilled weave and a 80×700 Dutch twilled weave.

Woven meshes can be a very efficient evaporative member (and also heat transfer member) due to their large effective surface area in comparison to their footprint area. Leveraging this insight, evaporative members in forms alternative to a mesh but have a relatively large effective surface area in comparison to their footprint area can be used in the dispensers of the present disclosure. By way of example only, evaporative members having an effective surface area of 2, 3, 4, 5, or more times greater than their respective footprint area are contemplated for use herein.

In one form, the mesh material can be fluid/liquid resistant for at least 50 days. The mesh material can have a workable life for at least two years in one embodiment. The mesh material can withstand a wide range of temperature changes; for example, from about −20° C. to about 150° C., or from about −10° C. to about 120° C.

Depending on the configuration of the fluid transfer member and its composition transfer surface, and the configuration of the evaporative member, the composition arriving from the fluid transfer member may be in a liquid state, a vapor state, or a mixed state. By way of example only, if a porous wicking element is used for the fluid transfer element and a mesh is employed for the evaporative member, the pore sizes of the respective components will tend to dictate the state of the composition within the mesh evaporative member. That is, if the pore size of a porous wicking element is smaller than a mesh pore size, then composition in liquid form may not move to the mesh, and instead will need to vaporize before passing into and through the mesh. Conversely, if the pore size of a mesh evaporative member is smaller than that of a porous wicking element, then capillary suction will tend to pull portions of the composition in liquid form into the mesh. In one form, primary composition transition from a liquid state to a vapor state will take place in the evaporative member, with optional secondary composition transition at the fluid transfer member's composition transfer surface.

Referring again to FIG. 1, a heating element 18 is proximate evaporative member 16. The heating element can raise the temperature of the evaporative member (and composition transfer surface) to a temperature of about 30° C. to about 150° C. In one form, heating element 18 contacts evaporative member 16 in at least one configuration. In another form, heating element 18 is affixed to evaporative member 16 via solder welding, mechanical fixing/pressing, or other appropriate technique. Heating element 18 shown in FIG. 1 is a ring-shaped heating element. FIG. 4 illustrates another heating element 40, that is disk-shaped, along with electrical leads 42 and 44 connected thereto. The heating element may be cylindrical-shaped or any other shape not shown in the figures. The heating element can be made from numerous conductive materials, including ceramic and metal. The heating element can have a footprint size similar to that of the composition transfer surface of the fluid transfer member. Heating elements having other shapes and sizes are contemplated herein. Besides a heating system, the dispensers may also include other evaporative assistance elements such as, for example, a fan or an agitation member. An optional fan associated with the dispenser may be operational at the same time as the heating element to help mitigate condensation.

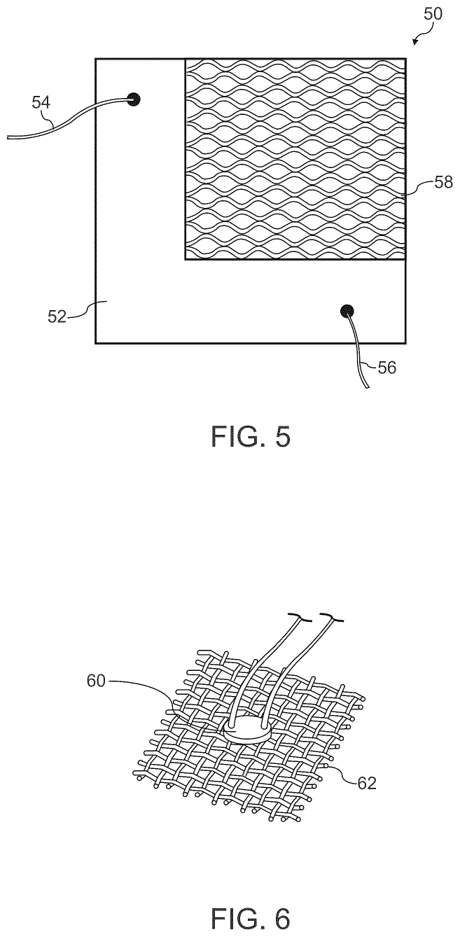

While FIG. 1 shows evaporative member 16 and heating element 18 as separate components, they can optionally be integrally formed so long as the electrical leads are attached to a portion of the same that provides enough electrical resistance to generate the necessary heat to cause a contained liquid composition to transition from a liquid state to a vapor state for emission from the dispenser. An example of an integrally formed evaporative member and heating element 50 is illustrated in FIG. 5 and comprises a heating element portion 52 with accompanying electrical leads 54 and 56, and an evaporative member portion 58 in the form of an expanded mesh.

The inventors have surprisingly discovered particularly useful ratios of the size of the heating element to the size of the evaporative member. A heating element footprint area to evaporative member footprint area ratio of from about 0.08 to about 1 are useful according to dispensers provided herein. With reference to FIG. 6, a circular-shaped disk heating element 60 having a diameter of about 6 millimeters to about 16 millimeters affixed to a 20 millimeters by 20 millimeters woven wire mesh 62 can provide energy-efficient dispensing results.

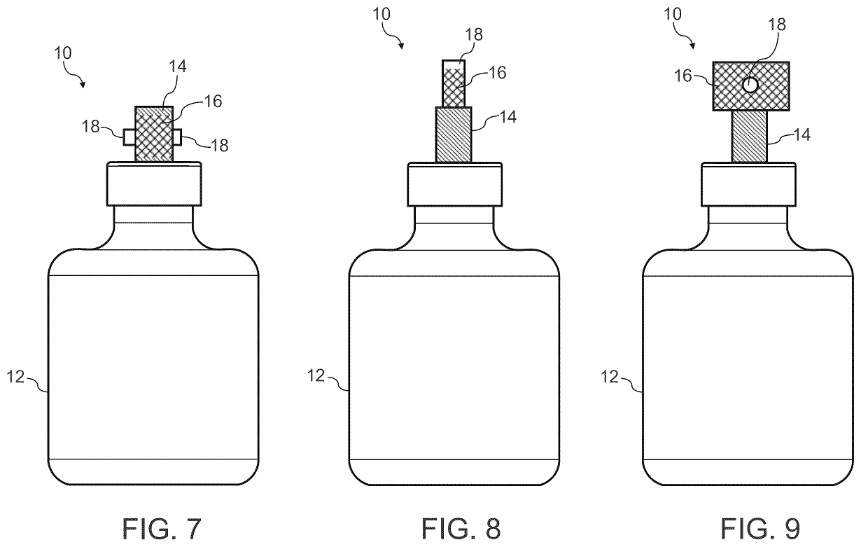

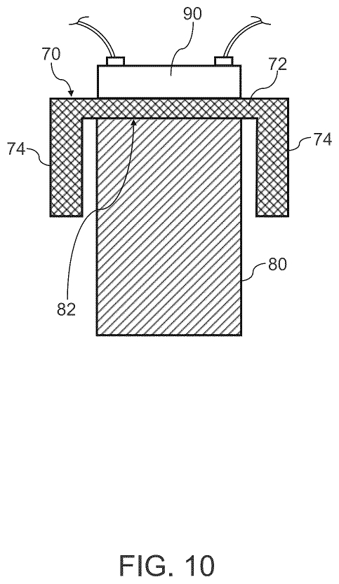

Alternative dispenser embodiments are shown in FIGS. 7 to 9, wherein like features are labeled with the same reference numerals as used in FIG. 1. In FIG. 7, the evaporative member 16 is wrapped around the fluid transfer member 14. Evaporative member 16 is heated with heating element 18. In FIG. 8, evaporative member 16 is wrapped around heating element 18, which is in direct contact with fluid transfer member 14. Evaporative member 16, as shown in FIGS. 7 and 8, may comprise a partial wrap, a full wrap, or multiple wrap layers. Referring now to FIG. 9, evaporative member 16 is vertically connected to fluid transfer member 14, with a heating element 18 being attached to a surface of evaporative member 16, which can have different configurations, such as, for example, flat, cylindrical, spiral, etc.

The mesh may be wrapped outside of the wick and the heater via the contact heater attached to the surface. The mesh can have a single layer or be folded in multiple layers.

Besides those shown and described above, the evaporative members may take on different configurations. By way of example only, an evaporative member 70 that is an upside down U-shape is shown in FIG. 10. In this configuration, a base portion 72 of evaporative member 70 is in contact with a composition transfer surface 82 of fluid transfer member 80. Side portions 74 of evaporative member 70 extend along part of fluid transfer 80, but with a small gap therebetween. Side portions 74 not only provide additional evaporative surface area, but they also create radiative heat transfer via heating element 90 to areas of the fluid transfer member close to its composition transfer surface 82, which can cause a more rapid or otherwise efficient transition of the volatile composition from a liquid state to a vapor state.

In some examples, the evaporative member 16 may be movable relative to the fluid transfer member 14. The evaporative member 16 may be fixed to the heating element 18 in an example where the heating element 18 is also movable relative to the fluid transfer member 14. In another example, the evaporative member 16 may move in and out of contact with the heating element 18 as well. The evaporative member 16 may be configured to move from a first position to at least a second position. In the first position, the evaporative member 16 is in contact with the fluid transfer member 14. While in the first position, a portion of the contained volatile liquid composition in the liquid state moves from the fluid transfer member to the evaporative member until the evaporative member reaches a maximum load or until the evaporative member is moved out of contact with the fluid transfer member. In the second position, the evaporative member 16 is spaced apart from the fluid transfer member 14 such that there is no contact. While in the second position, no portion of the contained volatile liquid composition in the liquid state moves from the fluid transfer member to the evaporative member.

The fluid transfer member 14 and evaporative member 16 may be configured in various arrangements. The evaporative member 16 may be positioned above a top of the fluid transfer member 14, and the device may be configured to move the evaporative member 16 up and down to contact the fluid transfer member 14. As another example, the evaporative member 16 may be positioned to a side of the fluid transfer member 14, and the device may be configured to move the evaporative member 16 sideways to contact the fluid transfer member 14.

When the evaporative member 16 contacts the fluid transfer member 14, a portion of the volatile composition begins to move from the fluid transfer member 14 to the evaporative member 16. For example, the evaporative member 16 may absorb the volatile composition from the fluid transfer member 14 through capillary action. The characteristics of the evaporative member 16 (e.g., weave pattern, area, and thickness) may be varied to control the rate at which the evaporative member 16 absorbs the volatile composition. The evaporative member 16 may then be moved out of contact with the fluid transfer member 14. The evaporative member 16 may be moved out of contact after a set duration of contact time or after a desired amount of volatile composition has moved onto the evaporative member 16 from the fluid transfer member 14. When the evaporative member 16 is in contact with the heating element 18 and not the fluid transfer member 14, the heating element 18 may be activated. Activating the heating element 18 will cause the volatile composition carried by the evaporative member 16 to evaporate. Evaporating the volatile composition primarily or entirely from the evaporative member 16 and not the fluid transfer member 14 can reduce buildup in the fluid transfer member 14. Heating the wick directly can result in disproportionate evaporation of higher vapor pressure components of the volatile composition relative to the lower vapor pressure components, which can result in changes over time of the delivered scent characteristic and delivery rates. Reducing buildup of higher vapor pressure components in the fluid transfer member 14 can improve the consistency of the scent characteristics and delivery rate of the evaporated volatile composition over the life of the product.

When all or a portion of the volatile composition carried by the evaporative member 16 has evaporated, the evaporative member 16 may be moved into contact with the fluid transfer member 14 again to load more volatile composition onto the evaporative member 16. If all of volatile composition carried by the evaporative member 16 is evaporated before contacting the fluid transfer member 14 again, the volume loaded onto the evaporative member 16 during each contact period can be more accurately controlled.

The desired or target volume of the volatile composition transferred during a single contact period may be from greater than 0 mg to about 100 mg, greater than 0 mg to about 1 mg, about 0.01 mg to about 50 mg, about 0.01 mg to about 10 mg, or about 0.01 mg to about 5 mg. The volatile composition dispenser may be configured to monitor or sense the amount of volatile composition that transferred to the evaporative member 16 during contact.

The distance that the evaporative member 16 travels may vary. For example, the distance of travel may be, for example, at least 1 mm, or at least 2 mm, or at least 3 mm, or at least 4 mm, or in a range of about 1 mm to about 10 mm.

The duration of the contact between the mesh and wick may vary. For example, a longer duration may allow the mesh to accumulate more volatile composition compared to a shorter contact duration. The duration may be configured to allow the evaporative member 16 to absorb a maximum amount of volatile composition, which may be based on the physical characteristics of the evaporative member 16. In another example, the duration may be timed such that the evaporative member 16 absorbs a certain percentage of the maximum amount. In an example where a specific evaporative member 16 takes 10 seconds of contact to absorb the maximum amount, the duration may be less than 10 seconds to absorb less than the maximum amount.

Different mechanisms may enable the movement of the evaporative member 16. For example, a motor system, electroactive or thermoactive material (e.g., in a spring form), magnetic attractions or repulsion, thermoactive gas/fluid mechanism, or a combination of the above. A mechanical biasing mechanism that tunes motion at temperature and/or motion at power threshold may be used. The motion may also facilitate cleaning of the interface and/or evaporative member 16 and further serve as a safety mechanism. For example, movement of the evaporative member 16 may induce a surface structure change and/or a change in temperature, which may dislodge or prevent accumulation of debris/deposition on the interface or evaporative member 16. In an example, the motion or change in geometric characteristics may limit the maximum load of available volatile composition that can be combusted in a thermal runaway condition. In an example, the evaporative member 16 may be constructed of a thermoactive material and exhibit characteristics of both an evaporative role and a motion role. The evaporative member 16 may be configured to interact with a dedicated cleaning mechanism during motion. For example, it's the movement may cause direct or indirect engagement with a cleaning element (e.g., wiper, brush, vibratory feature, secondary heating element, etc.).

In an example, the evaporative member may be configured to interact with more than one fluid transfer member, each fluid transfer member being in fluid communication with a distinct volatile liquid composition reservoir. For example, the evaporative member may be designed to sequentially and/or simultaneously engage with the various fluid transfer members to absorb the respective volatile compositions, which can then be evaporated upon activation of the heating element. The ability to dispense multiple volatile compositions from a single evaporative member can allow for dynamic scent profiles, improved consumer noticeability, and reduced habituation to individual scents over time. The volatile composition dispenser may be configured to alternate or mix the delivery of different volatile compositions based on user preferences or environmental conditions, thereby providing a customizable fragrance experience.

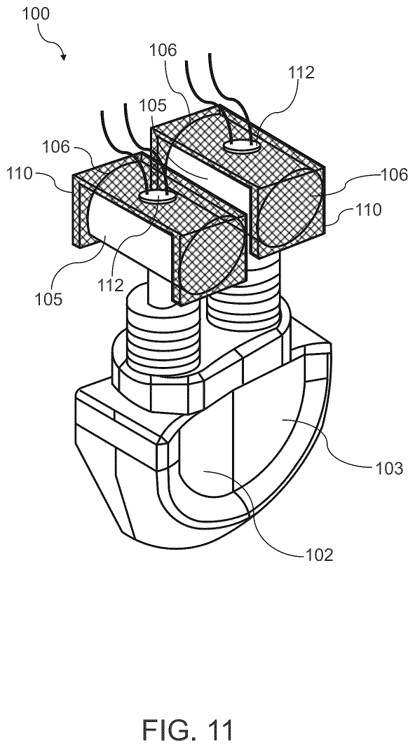

The dispensers can employ two or more reservoirs for holding different volatile liquid compositions. A dispenser that can emit multiple different compositions can improve noticeability of the volatilized composition and/or reduce the likelihood of short-term or long-term habituation of a single volatilized composition. An exemplary dispenser 100 that includes two separate volatile liquid composition reservoirs 102, 103 is shown in FIG. 11. A T-shaped fluid transfer member 105 is in fluid communication with each of reservoirs 102, 103. A fluid transfer member that has multiple composition transfer surfaces may be made as a single component or with multiple components that are fluidly connected to one another. Each of the T-shaped fluid transfer members 105 have two composition transfer end surfaces 106, which can produce better vapor dispensing profiles and/or improve the dispenser's energy efficiency. Alternatively, the entire exterior surface of the cylindrical portion of the fluid transfer member may act as a composition transfer surface instead of just the end regions. Upside down U-shaped meshes 110 are in contact with opposing composition transfer surfaces 106 of fluid transfer member 105. Individual heating elements 112 are affixed to the two meshes 110.

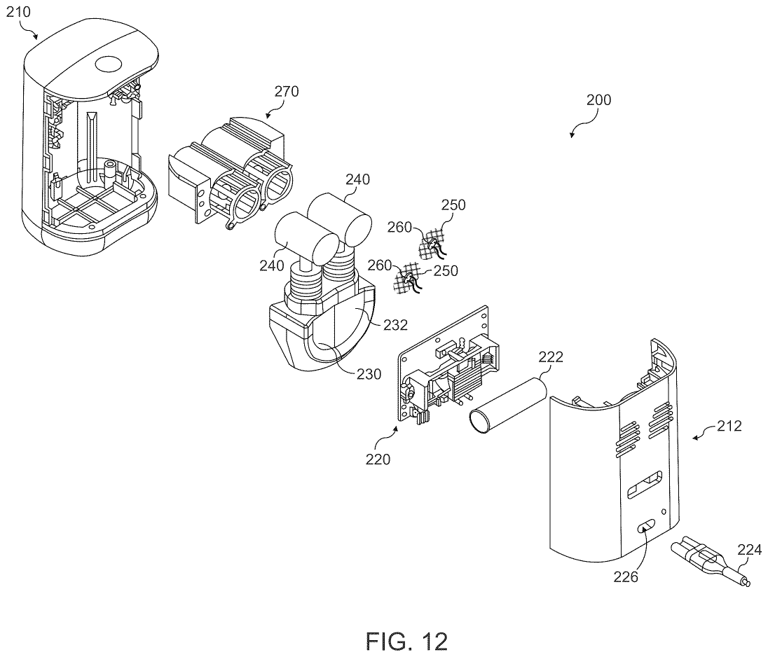

The dispenser components described above can be partially or fully contained within an optional housing. FIG. 12 is an exploded view of an exemplary dispenser 200 that includes a housing composed of a first or rear housing shell 210 and a second or front housing shell 212, which can be removably connected to one another. An electrical assembly 220 and rechargeable battery 222 (e.g., chargeable by a cable 224 via charging port 226) are included to provide power and control aspects of dispenser 200. Two separate volatile compositions can be stored and delivered via the individual reservoirs 230, 232, along with fluid transfer members 240, evaporative members 250, and heating elements 260. A mounting member 270 assists a user with changing the volatile composition reservoirs when depleted or when a user may otherwise choose to make a change in what is being dispensed.

In FIGS. 11 and 12, each of the two composition reservoirs has its own dedicated heating and evaporation systems. This design approach can enable control flexibility. However, alternative configurations are possible when a dispenser employes multiple volatile liquid composition reservoirs. For example, a single heating element can be employed with two or more individual evaporative members that are in direct contact with the separate fluid transfer members. Also, a single evaporative member can span the two fluid transfer members and be heated by multiple, individual heating elements. When heating is directed primarily to one of the evaporative members, there can be some heating of the liquid composition in adjacent evaporative members. This can improve the speed and/or efficiency of vaporizing a liquid composition in the adjacent evaporative member when the primary heating is switched to the same since the liquid composition is already in a warmed state prior to the switching.

The volatile composition dispenser can comprise a number of additional optional features. The volatile composition dispenser can be provided with indicators so that a person is further made aware that the volatile material being emitted has changed. Such indicators can be visual and/or audible, such as lights or sounds, respectively. For example, in the case of scented materials, such an indicator may allow a person to see which scent is being emitted at a given time. In another example, at least a portion of the volatile composition dispenser (such as all or a portion of the optional housing) or the reservoirs may be made of a type of plastic that changes color when heated.

The volatile composition dispenser can be provided with additional user controls. The volatile composition dispenser can include a power switch to allow a user to turn the volatile composition dispenser ON and OFF without removing it from an electrical socket or other power source. The volatile composition dispenser can be provided with a control that allows the user to control the discrete emission period of one or more of the volatile compositions, and/or the time between the emission of the different volatile compositions, or the time that the volatile materials are emitted during a simultaneous operation period. For example, in one non-limiting example, if the volatile composition dispenser is provided with the capability of emitting each volatile material during a period greater than 15 minutes and less than or equal to 48 hours, then the volatile composition dispenser can be provided with a control that allows the user to set the discrete emission period for one or more of the volatile compositions to 30 minutes, 45 minutes, or 72 minutes, or to one hour, for example.

The volatile composition dispenser can comprise a thermostat or other switch to allow a user to adjust the temperature settings of the heat sources for one or more of the volatile compositions. The settings may be predefined for particular volatile compositions, or may be adjustable based on selected temperatures to be applied to a wick. The settings may include a LOW and HIGH settings or LOW, MEDIUM, and HIGH settings, for example, that a user can set either directly on the volatile composition dispenser or remotely through a remote control (e.g., computer, phone, etc.). A device may have one, two, three, four, five, six, or more different intensity settings. The settings may be labeled as an intensity (i.e., HIGH, MEDIUM, LOW, etc.) or room-type (i.e., bathroom, bedroom, living, kitchen, etc.).

The volatile composition dispenser may also include sensors and the volatile composition dispenser may be programmed to adjust for the readings of the sensors. For example, the volatile composition dispenser may include sensors such as temperature sensors, relative humidity sensors, volatile material sensors, light sensors (e.g., detecting day/night), sensors to detect the fill level of the composition reservoir, and the like.

The volatile composition dispenser may be communicably connectable with various components of the dispenser, including the sensor(s), heating elements, user interface, etc., using a wireless communication link. Various wireless communication links may be used, including 802.11 (Wi-Fi), 802.15.4 (ZigBee, 6LoWPAN, Thread, JennetIP), Bluetooth, combinations thereof, and the like. Connection may be through an ad hoc Mesh Network protocol. The controller may include a wireless communication module in order to establish a wireless communication link with the controller with various components of the system. Any module known in the art for establishing such communication links can be utilized. The controller may utilize a machine learning algorithm, such as a NEST® learning thermostat.

The reservoir may include an identification tag, such as an RFID tag and the optional housing of the volatile composition dispenser may include an RFID tag reader. An RFID tag may be used to tell the controller details about the volatile composition contained in the reservoir, such as the scent. The volatile composition dispenser may include programs that adjust to account for information read from the RFID tag.

The volatile composition dispenser may include a tactile switch or registration point that, upon coming in contact with a reservoir, provides signals to the volatile composition dispenser including, but not limited to, a new or refilled reservoir that is full of a volatile composition has been inserted, an old cartridge has been removed, etc. An included printed circuit board (PCB) could interpret these signals and cause the volatile composition dispenser to act to programmed instructions accordingly, such as starting the total emission program for a new or refilled composition reservoir that is “full” of a volatile composition.

The volatile composition dispenser can also be sold in the form of a kit that includes the volatile composition dispenser and one or more replacement reservoirs of volatile compositions. The volatile composition dispenser and/or kit can also include instructions for use that instruct the user regarding certain discrete emission periods that may be used to produce certain results, and/or instructions regarding where to place the volatile composition dispenser in a given space. For example, the instructions may include instructions for setting the volatile composition dispenser based on the size of the room, vehicle, etc. in which the volatile composition dispenser is placed. Such instructions may also include instructions to the user to choose more frequent changes between the emissions of scented materials for greater scent awareness. Instructions may also be provided to specify how to operate the volatile composition dispenser relative to other volatile composition dispensers. The instructions can be provided in any suitable form (e.g., written, audio, and/or video).

The volatile composition dispenser may include a power source, such as a plug or battery. The volatile composition dispenser may be battery powered so that it need not be plugged into an electrical outlet. If a plug is used as the power source to connect to an electrical outlet, the plug may include a cord or may be a wall-mount plug. The volatile composition dispenser can also be configured so that it can be both plugged in and powered by a source of electrical current, and also battery powered. The volatile composition dispenser can also be provided with an adapter so that it can be plugged into the cigarette lighter in a vehicle. In addition, the volatile composition dispenser can be provided with a remote control that allows the user to control any, or all, of the emission properties of the volatile composition dispenser (including, but not limited to, changing the volatile material being emitted) without touching the volatile composition dispenser.

The volatile composition dispenser may comprise a microprocessor that has fewer component parts compared to analog circuits, and improved circuit quality from lot to lot. The microprocessor can allow the user to program and control the temperature profile by modulation to alter performance. If desired, the microprocessor may be connected to a user interface. This can be any suitable type of user interface. Examples of types of user interfaces include, but are not limited to, LCD screens and LEDs, buttons (e.g., push buttons or buttons that move side-to-side), dials, and the like. In addition, the microprocessor enables components to allow multiple volatile composition dispensers (e.g., those located in different parts of a room, or in different rooms), to communicate with each other. For example, the microprocessor can enable a remote control to send digital signals via an infrared beam to turn another volatile composition dispenser ON or OFF.

The heating elements may be programmed to operate in various operational conditions. As will be discussed in more detail below, the heating elements may be configured to enable various discrete emission periods, gaps in emission of any heating elements, varying energy profiles over time, randomized energy profiles, simultaneous emission periods, and combinations thereof. Each of these methods of operation, either alone or in combination, may promote user noticeability of the volatile composition and/or reduce the likelihood of short-term or long-term habituation of the volatile composition.

The term “discrete emission period,” as used herein, refers to the individual time period that a given volatile composition is emitted in an emission sequence. This may correspond generally to the period of time that a heating element is turned ON for a given fill of volatile composition, although there may be a slight lag between the operation of a heating element and the emission of a volatile composition. The term “extended emission periods”, as used herein, includes a plurality of successive discrete emission periods that may be separated by gaps in operation where the heating element is OFF.

The “total emission program” refers to the entire sequence, including all discrete emission periods and OFF times for gaps in emission that make up the energy boosts and extended emission periods, from beginning to end of life of a “filled” volume of volatile composition in a reservoir. “Fill” or “filled,” as used herein, refers to an amount of volatile composition is intended to occupy the whole of or substantially the whole of the available volume in the reservoir, which excludes any volume occupied by any other elements of the volatile composition dispenser that may be disposed in the reservoir, such as the evaporative member. The reservoir will typically be occupied or filled to at least 80%, 85%, 90%, or 95% volatile composition, of the total available volume of the reservoir. The total emission program is then designed to evaporate all or substantially all of the volatile composition in the reservoir.

The total emission program may be continuous. The term “continuous,” as used in reference to the emission program, means that there is a planned emission sequence over an entire period, once the program is initiated. This emission program can include periods, as noted above, where there are gaps in emission. This will still be considered to be a continuous emission program, although there will not necessarily be continuous emission of volatile compositions. It should be understood, however, that it is possible for the emission program to be interruptible by the user (e.g., turned off), if desired. The emission program may be designed to run continuously, or substantially continuously until at least one of the volatile compositions is substantially depleted from the reservoir. It may be desirable for the emission program to run continuously until all of the volatile compositions are substantially depleted, and for this to occur at approximately the same time.

If the total emission program is disrupted, the dispenser may be configured with memory to record the last emission sequence that was initiated in the event that the volatile composition dispenser is disconnected from the power source. Once operation of the volatile composition dispenser is resumed, the memory of the last recorded sequence is recalled to return the total emission program to the correct emission sequence. The total emission program may only be restarted at the beginning of the program when a new or refilled reservoir is used.

The total emission program can be of any suitable length, including but not limited to 10 days, preferably 15 days, preferably 20 days, preferably 25 days, preferably 30 days, more preferably 45 days, more preferably 60 days, more preferably 90 days, more preferably 130 days, more preferably 150 days, or shorter or longer periods, or any period between 30 to 150 days.

The discrete emission period for each evaporative member in a volatile composition dispenser may be in the range of 2 minutes to 48 hours, alternatively 5 minutes to 48 hours, alternatively 10 minutes to 48 hours, alternatively 15 minutes to 48 hours, alternatively 20 minutes to 24 hours, alternatively 30 minutes to 8 hours, alternatively 45 minutes to 4 hours. The higher the energy supplied by the heating element, the shorter the discrete emission period that may be needed to provide a noticeable amount of volatile composition into the air.

During the discrete emission period for a particular evaporative member, the heating element will be continuously ON. In a volatile composition dispenser comprising more than one heating element, the heating elements may have alternating discrete emission periods. In an alternating system, one heating element may be turned ON while the other heating element(s) may be turned OFF. Or one or more heating elements may be turned ON at a given time. The operation of two or more heating elements may overlap for a period of time. The greater the discrete emission period for each evaporative member, the potential for higher concentrations of volatile composition in the surrounding space in order to increase user noticeability. There may also be time periods when all heating elements are turned OFF. Each evaporative member may be configured to have the same discrete emission period, or some or all of the evaporative members may be configured to have different discrete emission periods.

Evaporation rates of the volatile composition from the evaporative member may be between 5 mg/hr and 200 mg/hr, preferably between 10 mg/hr and 100 mg/hr, more preferably between 10 mg/hr and 80 mg/hr, more preferably between 15 mg/hr and 60 mg/hr, and more preferably between 15 mg/hr and 50 mg/hr, and more preferably 15 mg/hr to 35 mg/hr over the total emission program.

Near the end of the total emission program, the volatile composition dispenser may operate at or near the maximum power output, such as maximum temperature, until unplugged and a new composition reservoir is used.

The total emission program may be configured to turn OFF a heating element when the volatile composition is depleted from the reservoir. For example, the heating element may turn OFF after a predetermined time period for a given intensity setting. By turning OFF the heating element, energy is not applied by the heating element until the reservoir is refilled or replaced with a new fill of volatile composition.

Varying the energy applied by the evaporative member over the total emission program may improve consumer noticeability of the volatile composition and help prevent habituation of the volatile composition. In order to increase noticeability of the volatile composition evaporated from the volatile composition dispenser and prevent noticeability from continually declining over the life of the volatile composition in the volatile composition dispenser, the evaporation rates may be constant, substantially constant, increasing, or variable. In order to achieve constant, substantially constant, increasing, or variable evaporation rates, the energy applied to the evaporative member by the heating element can be varied to achieve the desired evaporation profile over the total emission program. For example, in order to deliver a constant, substantially constant, or even increasing evaporation rate over time, the power to the heating element and/or the ON-time of the heating element can be continually increased over time. In order to achieve an increasing evaporation rate over time, the power applied by the heating element and/or the ON-time of the heating element may need to be greater than the power applied to, and/or the ON-time of, the heating element as compared to the operation of a heating element programmed to maintained a constant or substantially constant evaporation rate. In order to create a random or variable evaporation rate over the total emission cycle, the power applied to the heating element and/or the ON-time of the heating element can be increased, maintained, and/or decreased over time. The power applied to the heating element may be adjusted at a variety of frequencies.

The energy applied to the evaporative member can either be increased, decreased, or maintained at any given point within the total emission program. It has been found that a total emission program having a combination of extended emission periods of increased energy (“energy boost”), decreased energy, and/or maintained energy provides improved consumer acceptance of a volatile composition dispenser over commercially available volatile composition dispensers.

It has been found that consumers expect a minimum level of noticeability of the volatile composition at the beginning of life of a composition reservoir. A volatile composition dispenser that meets this expectation at the beginning of life can actually improve consumer acceptance of the volatile composition dispenser not only at the beginning of life, but for the total emission program. As such, an energy boost period of a relatively high energy at the beginning of the total emission program to meet or exceed the consumer's minimum level of noticeability requirement may be desirable. Thus, an initial energy boost period applied to the evaporative member within the first 24 hours of operation of the total emission program of the volatile composition dispenser should be sufficiently high to meet or exceed the consumer's minimum desired evaporation rate for the volatile composition.

An energy boost at various extended emission periods over the life of the volatile composition in the reservoir can increase noticeability of the volatile composition over the total emission program by maintaining or increasing the evaporation rate of the volatile composition from the evaporative member over time.

The total emission program may also include extended emission periods of decreased energy applied to the evaporative surface. Decreasing the energy applied to the evaporative member for periods of time can conserve the volatile composition such that the volatile composition may extend the total time of the total emission program. Decreasing the energy applied to the evaporative member can also improve noticeability over time because a subsequent energy boost may result in a bigger change in energy over the same time period.

The total emission program may also include extended emission periods of maintained energy applied to the evaporative surface. Maintaining the energy applied to the evaporative member in between periods of an energy boost can conserve the volatile composition such that the volatile composition may deplete from the composition reservoir slower. Applying an energy boost more frequently than is needed to increase consumer noticeability may be volatilizing more volatile composition than is necessary.

The extended emission periods may include periods of decreasing energy, maintained energy, relatively small increases in energy that remain below the energy of the previous or subsequent energy boost, or combinations thereof.

The length of an energy boost period may extend for up to half of the length of the extended emission periods of decreasing and/or maintaining energy. Or length of an energy boost may extend for up to one-third of the length of the extended emission periods of decreasing and/or maintaining energy. An energy boost may occur on a daily, weekly, or biweekly basis with extended emission periods in between. One energy boost may be at a temperature in the range of about 40° C. to about 80° C., wherein a subsequent energy boost may be at a temperature in the range of about 50° C. to about 90° C., and a subsequent energy boost may be at a temperature in the range of about 60° C. to about 100° C.

In a configuration wherein the energy is adjusted through the length of time of the discrete emission periods for the heating element(s), one energy boost may have a discrete emission period(s) of 20 minutes to 90 minutes, a subsequent energy boost may have a discrete emission period(s) of 40 minutes to 110 minutes, and a subsequent energy boost may have a discrete emission period(s) of 60 minutes to 130 minutes.

An energy boost period may increase the energy by at least 5%, or at least 10%, or at least 15%, or at least 20%, or at least 30%, or at least 40%, or at least 50%, or at least 60%, or at least 75%, or at least 100%, or at least 150%, or at least 200% of the energy applied immediately before the energy boost period. The greater the increase in energy at an energy boost period, the more noticeable the volatile composition may be to the consumer during and following the energy boost period. Successive energy boost periods in a total emission program may increase by a greater percentage than the previous energy boost periods in order to achieve the desired uniform or increased evaporation rate.

An energy boost period may increase the evaporation rate by at least 5%, or at least 10%, or at least 15%, or at least 20%, or at least 30%, or at least 40%, or at least 50%, or at least 60%, or at least 75%, or at least 100%, or at least 150%, or at least 200% of the evaporation rate before the energy boost period.

Energy boost period may occur over a length of time that is shorter than the length of time of an extended emission period in order to extend the life of the volatile composition in the reservoir. As such, the length of time of an energy boost period may be no more than half of the length of an extended emission period, or the length of an energy boost period may be no more than one third of the length of an extended emission period. An energy boost period may occur over the course of 1 day and an extended emission period may occur over the course of 2 to 6 days, for example. Or an energy boost may occur over the course of 2 days and an extended emission period may occur over the course of 4 to 6 days, for example.

Throughout the detailed description and claims, the energy boosts may be referred to as “a first energy boost,” a “second energy boost,” “a third energy boost,” etc. It is to be understood that the number used with the term “energy boost” is used only to different between different energy boosts and is not intended to limit the order in which the energy boosts occur. That is, an additional energy boost or multiple energy boosts may occur between two sequentially numbered energy boosts. For example, “second energy boost” and a “third energy boost” may be separated by an additional one or more energy boosts.

The total emission program may include gap periods where all heating elements of a volatile composition dispenser are turned OFF (“gap periods”). By introducing gap periods, the noticeability of the volatile composition in the space declines so that a user is less likely to be become habituated to the volatile composition.

Another method that may be used to achieve comparable results is to decrease the energy of the heating element periodically such that the energy provided to the evaporative member is below that which is necessary to evaporate the volatile composition at a sufficient level for the odor detection threshold (“ODT”) of the volatile composition. The lowered evaporation rate and operation at sub-ODT levels provides a break from sensory stimulus, thus enabling enhanced noticeability when the stimulus is reintroduced. This operation also maintains the evaporative member at a heightened state above ambient conditions enabling faster response time and/or less energy in order to return to steady-state upon the initiation of the next cycle phase.

Operating below the ODT of a volatile composition may translate to operating the heating element at less than 30% of maximum power output, or less than 25% of maximum power output, or less than 20% of maximum power output, or less than 15% of maximum power output, or less than 10% of maximum power output.

There are many different approaches for programming the heating elements to operate in the various configurations discussed above. As a non-limiting example, where a volatile composition dispenser includes two heating elements, Heating element A and Heating element B, Heating element B OFF time is equal to the ON time of Heating element A operation and occurring either in parallel (e.g., Heating element B is simultaneously OFF while Heating element A is operating) or in series (e.g., Heating element A is ON for 30 mins and then Heating element B is OFF for 30 mins). The OFF time of each heating element may be in the time period of 0 mins to 48 hours, preferably 5 mins to 24 hours, and more preferably 10 mins to 24 hours.

The total emission program may include simultaneously operating the one or more heating elements. The heating elements can be operated simultaneously at a plurality of energy levels to create new volatile composition experiences that are more noticeable. By combining multiple volatile compositions and varying the evaporation rates of said volatile compositions, it is possible to create continually changing experiences (both in terms of intensity and in character) that combat both habituation. Having a plurality of volatile compositions over time allows for a unique and ever-changing stimulus that may always be noticeable. Operating a plurality of volatile composition simultaneously may also enable higher evaporation rates than are achievable by utilizing a single volatile composition while conserving total system volatile composition amounts. The heating elements, if operated simultaneously, can operate simultaneously for just a portion of operation of each heating element. For example, one heating element may run on at one time, followed by a period where the heating element may run simultaneously with one or more of the other heating elements, optionally followed by a period where the first heating element to run may be turned OFF and the one or more other heating element(s) may run on its own.

### Method of Measuring the Wicking Time of an Evaporative Member in the Form of a Mesh

Gloves should be worn when handling the mesh sheets and cut samples as oils from the skin could affect results. For each sheet of mesh sample to be tested, label one side as the ‘X Axis’ and the other side (90 degree turn) as the ‘Y-Axis’ side. Cut three samples to 7 cm×1 cm size (other sample sizes can be chosen depending on the design) from the X-Axis side and the Y-Axis side (i.e., there will be 3 samples with the 7 cm cut length from the X-Axis side of the mesh and 3 samples with the 7 cm cut length from the Y-Axis side of the mesh). Mark a line 3 cm above the bottom edge of the longer cut side (7 cm). In a 50 ml beaker, add 5 g of a composition perfume. The perfume composition used for the testing is non-aqueous and contains at least 10% dipropylene glycol methyl ether solvent and has a viscosity of approximately 4.8 cPs and a density of approximately 0.92 g/ml. A suitable example of a perfume composition for this testing is from the Febreze PLUG™ Linen & Sky refill. Suspend the mesh sample with the 7 cm cut side vertically positioned above the perfume composition, and confirm that the sample is hanging level. Lower the mesh sample so that the bottom edge touches the top surface of the liquid perfume composition. The mesh sample should be lowered no more than 1-2 mm into the liquid. Start the timer as soon as the mesh is in contact with the perfume composition, and record the time for the perfume composition to wick upward to the 3 cm line. The testing is repeated with each of the 3 samples cut from the X-Axis side and each of the 3 samples cut from the Y-Axis side of the mesh. For each mesh material, the wicking time reported is the average wicking time across the 6 samples. Any mesh with at least one sample that does not wick to the 3 cm line after 30 minutes does not receive an average wicking time and is marked as not wicking out. All testing is conducted at room temperature 21° C. (+/−2° C.). A small amount of an oil-soluble dye can optionally be added to the perfume composition to make it easier to visualize the perfume composition wicking out across the mesh.

### EXAMPLES

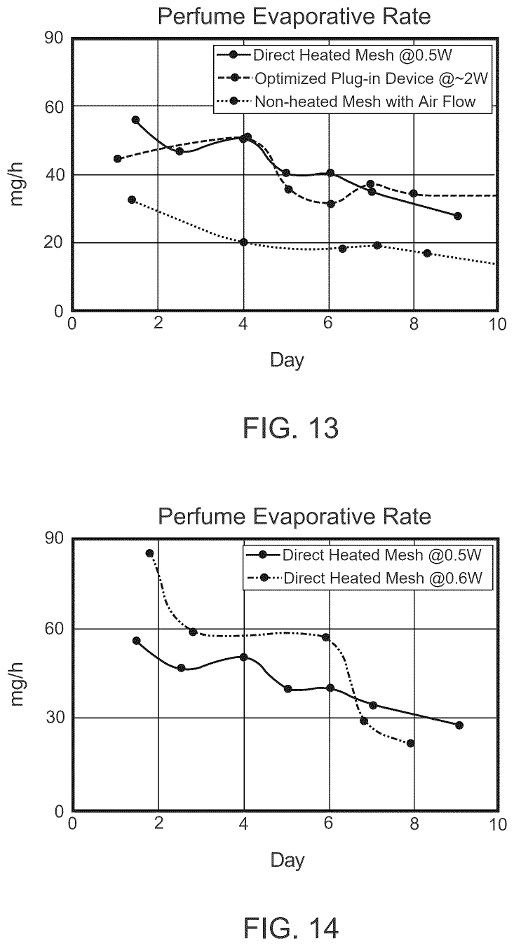

A dispenser similar to what is shown in FIG. 1 was assembled and the volatile composition reservoir filled with a perfume composition. A Dutch twilled weave 165×1,400 mesh having a footprint of 20×20 millimeters was used for the evaporative member. A ring ceramic heating element having an inner diameter of 3 millimeters and an outer diameter of 7 millimeters was affixed to the mesh. FIG. 13 shows the perfume delivery results from the exemplary dispenser with 0.5 W power input. This is compared with two other arrangements—one is a prior art dispenser with an approximate 2.0 W power input, and the other is a dispenser comprising a non-heated mesh evaporative surface exposed to an airflow of about 1 m/s every 30 minutes. As seen in FIG. 13, the exemplary dispenser can deliver equivalent or even enhanced evaporation of volatile compositions with a much lower energy input.

FIG. 14 shows perfume delivery results from an exemplary dispenser according to the description herein with two different power inputs-one at 0.5 W, and the other at 0.6 W.

It should be understood that the claims should not be limited to dispensers that provide the results as shown in FIGS. 13 and 14 because there are many variations of the inventive dispensers according to the description above, as well as a plethora of different volatile compositions useable in the dispensers that will dictate delivery rates, profiles, etc.

In another experiment, a dispenser similar to what is shown in FIG. 1 was assembled but the volatile composition reservoir was left empty. The heating element and the 20 mm×20 mm mesh achieved a steady state temperature of 137° C. in about 1 minute using a power input of 0.65 W. The wick temperature increased by about 25° C. within that first minute. When the power input was stopped, the heater and the mesh temperature decreased to room temperature in about 2 minutes. This is in contrast to some commercially available volatile dispensers, where it can take 15 to 30 minutes to achieve a target temperature and at least 15 minutes to cool back to an ambient state after power is turned off.

### Exemplary Dispenser Embodiments

A. A dispenser comprising:
a. a volatile liquid composition reservoir;
b. a fluid transfer member in fluid communication with the volatile liquid composition reservoir, and comprising a composition transfer surface capable of releasing some of a contained volatile liquid composition in a liquid state and/or vapor state;
c. an evaporative member in contact with the composition transfer surface; and
d. a heating element affixed to the evaporative member, wherein heat is conductively transferred to both the evaporative member and the composition transfer surface.

B. The dispenser as disclosed in A, wherein the evaporative member comprises a mesh.

C. The dispenser as disclosed in B, wherein the mesh comprises a metal wire mesh.

D. The dispenser as disclosed in B or C, wherein the mesh is a woven mesh.

E. The dispenser as disclosed in any one of B-D, wherein the mesh comprises from about 40 to about 500 openings per inch as measured in a first direction and from about 500 to about 3,000 openings per inch as measured in a second direction orthogonal to the first direction.

F. The dispenser as disclosed in any one of B-E, wherein the mesh comprises a weave pattern selected from the group comprising plain Dutch weave, reverse plain Dutch weave, Dutch twilled weave, and reverse Dutch twilled weave.

G. The dispenser as disclosed in any one of A-F, wherein the heating element comprises a ring-shaped or disk-shaped heating element comprising two electrical leads soldered thereto.

H. The dispenser as disclosed in any one of A-G, further comprising a battery.

I. The dispenser as disclosed in any one of A-G, further comprising a rechargeable battery.

J. The dispenser as disclosed in I, wherein the dispenser can continuously or discontinuously deliver a volatilized composition for a period of at least 12 hours.

K. The dispenser as disclosed in any one of A-J, wherein the dispenser can deliver dipropylene methyl ether at a rate of more than 75 mg/h when the heating element receives a power input of less than 1 W.

L. The dispenser as disclosed in any one of A-K, wherein the evaporative member can reach 95° C. or higher within 1 minute of the heating element receiving power input.

M. The dispenser as disclosed in any one of A-L, further comprising a fan that operates during which time power is delivered to the heating element.

N. The dispenser as disclosed in any one of A-M, further comprising:
e. a second volatile liquid composition reservoir;
f. a second fluid transfer member in fluid communication with the second volatile liquid composition reservoir, and comprising a second composition transfer surface capable of releasing some of a contained volatile liquid composition in a liquid state and/or vapor state;
g. a second evaporative member in contact with the second composition transfer surface; and
h. a second heating element affixed to the second evaporative member, wherein heat is conductively transferred to both the second evaporative member and the second composition transfer surface; and
wherein the dispenser is connectable to a power source and wherein power can selectively be directed to the first heating element, the second heating element, or directed to both of the first heating element and the second heating element.

O. The dispenser as disclosed in N, wherein a selected power input value to the first and second heating element can be programmed and stored on the dispenser.

P. The dispenser as disclosed in N or O, wherein the stored value can be sent to the dispenser wirelessly.

Q. The dispenser as disclosed in any one of N-P, further comprising a volatile liquid composition disposed in the volatile composition reservoir, and wherein some of the volatile liquid composition is released from the both the composition transfer surface and evaporative member in a vapor state.

R. A dispenser comprising:
a. a volatile liquid composition reservoir;
b. a fluid transfer member in fluid communication with the volatile liquid composition reservoir, and comprising a composition transfer surface; and
c. an evaporative member in contact with the composition transfer surface, the evaporative member comprising a mesh; and
d. two electrical leads connectable to a power source;
e. a heating element for transferring heat to the evaporative member, wherein the two electrical leads are connected to the heating element and not the mesh.

S. The dispenser as disclosed in R, wherein the mesh comprises a metal wire mesh.

T. The dispenser as disclosed in R or S, wherein the mesh is a woven mesh.

U. The dispenser as disclosed in any one of R-T, wherein the mesh comprises from about 40 to about 500 openings per inch as measured in a first direction and from about 500 to about 3,000 openings per inch as measured in a second direction orthogonal to the first direction.

V. The dispenser as disclosed in any one of R-U, wherein the mesh comprises a weave pattern selected from the group comprising plain Dutch weave, reverse plain Dutch weave, Dutch twilled weave, and reverse Dutch twilled weave.

W. The dispenser as disclosed in any one of R-V, wherein the heating element comprises a ring-shaped or disk-shaped heating element.

X. The dispenser as disclosed in any one of R-W, further comprising a battery.

Y. The dispenser as disclosed in any one of R-W, further comprising a rechargeable battery.

Z. The dispenser as disclosed in Y, wherein the dispenser can continuously or discontinuously deliver a volatilized composition for a period of at least 12 hours.

AA. The dispenser as disclosed in any one of R-Z, wherein the dispenser can deliver dipropylene methyl ether at a rate of more than 75 mg/h when the heating element receives a power input of less than 1 W.

BB. The dispenser as disclosed in any one of R-AA, wherein the evaporative member can reach 95° C. or higher within 1 minute of the heating element receiving power input.

CC. The dispenser as disclosed in any one of R-BB, further comprising:
e. a second volatile liquid composition reservoir;
f. a second fluid transfer member in fluid communication with the second volatile liquid composition reservoir, and comprising a second composition transfer surface capable of releasing some of a contained volatile liquid composition in a liquid state and/or vapor state;
g. a second evaporative member in contact with the second composition transfer surface; and
h. a second heating element affixed to the second evaporative member, wherein heat is conductively transferred to both the second evaporative member and the second composition transfer surface,
wherein the dispenser is connectable to a power source and wherein power can selectively be directed to the first heating element, the second heating element, or directed to both of the first heating element and the second heating element.

DD. The dispenser as disclosed in any one of R-CC, further comprising a volatile liquid composition disposed in the volatile composition reservoir, and wherein some of the volatile liquid composition is released from the both the composition transfer surface and evaporative member in a vapor state.

EE. A dispenser comprising:
a. a housing;
b. a volatile liquid composition reservoir;
c. a fluid transfer member in fluid communication with the volatile liquid composition reservoir, and comprising a composition transfer surface;
d. an evaporative member in contact with the composition transfer surface, the evaporative member comprising a mesh; and
e. a heating element for transferring heat to the evaporative member,
wherein the volatile liquid composition reservoir and fluid transfer member are removably contained within the housing and replaceable with another volatile liquid composition reservoir and fluid transfer member; and
wherein the evaporative member and the heating element are permanently contained within the housing.

FF The dispenser as disclosed in EE, wherein the mesh comprises a metal wire mesh.

GG. The dispenser as disclosed in EE or FF, wherein the mesh is a woven mesh.

HH. The dispenser as disclosed in any one of EE-GG, wherein the mesh comprises from about 40 to about 500 openings per inch as measured in a first direction and from about 500 to about 3,000 openings per inch as measured in a second direction orthogonal to the first direction.

II. The dispenser as disclosed in any one of EE-HH, wherein the mesh comprises a weave pattern selected from the group comprising plain Dutch weave, reverse plain Dutch weave, Dutch twilled weave, and reverse Dutch twilled weave.

JJ. The dispenser as disclosed in any one of EE-II, wherein the heating element comprises a ring-shaped or disk-shaped heating element.

KK. The dispenser as disclosed in any one of EE-JJ, further comprising a battery.

LL. The dispenser as disclosed in any one of EE-JJ, further comprising a rechargeable battery.

MM. The dispenser as disclosed in LL, wherein the dispenser can continuously or discontinuously deliver a volatilized composition for a period of at least 12 hours.

NN. The dispenser as disclosed in any one of EE-MM, wherein the dispenser can deliver dipropylene methyl ether at a rate of more than 75 mg/h when the heating element receives a power input of less than 1 W.

OO. The dispenser as disclosed in any one of EE-NN, wherein the evaporative member can reach 95° C. or higher within 1 minute of the heating element receiving power input.

PP. The dispenser as disclosed in any one of EE-OO, further comprising a volatile liquid composition disposed in the volatile composition reservoir, and wherein some of the volatile liquid composition is released from the both the composition transfer surface and evaporative member in a vapor state.

QQ. A dispenser comprising:
a. a volatile liquid composition reservoir;
b. a fluid transfer member in fluid communication with the volatile liquid composition reservoir, and comprising a composition transfer surface;
c. an evaporative member in contact with the composition transfer surface; and
d. a heating element for transferring heat to the evaporative member, the evaporative member comprising a mesh,
wherein the heating element has a higher electrical resistance than the mesh at a given electrical current.

RR. A dispenser comprising:
a. a volatile liquid composition reservoir;
b. a fluid transfer member in fluid communication with the volatile liquid composition reservoir, and comprising a composition transfer surface;
c. an evaporative member in contact with the composition transfer surface; and
d. a heating element for transferring heat to the evaporative member,
wherein the heating element comprises a first footprint area, wherein the evaporative member comprises a second footprint area, and wherein a ratio of the first footprint area to second footprint area is from about 0.08 to about 1.

SS. A dispenser comprising:
a. a volatile liquid composition reservoir;
b. a fluid transfer member in fluid communication with the volatile liquid composition reservoir, and comprising a composition transfer surface;
c. an evaporative member in contact with the composition transfer surface; and
d. a heating element for transferring heat to the evaporative member;
wherein the dispenser can deliver dipropylene methyl ether at a rate of more than 75 mg/h when the heating element receives a power input of less than 1 W.

TT. The dispenser as disclosed in any one of A-SS, wherein the fluid transfer member comprises a porous wicking element.

UU. The dispenser as disclosed in TT, wherein the porous wicking element comprises sintered polymeric particles.

VV. The dispenser as disclosed in any one of A-UU, wherein the heating element is positioned proximate to the evaporative member.

WW. The dispenser as disclosed in any one of A-UU, wherein the heating element is affixed to the evaporative member.

XX. The dispenser as disclosed in any one of A-VV, wherein portions of a contained volatile composition are transferred from the composition transfer surface to the evaporative member in both a liquid state and a vapor state.

YY. The dispenser as disclosed in any one of A-WW, wherein the evaporative member is in contact with the composition transfer surface in a first position, and the evaporative member is movable to a second position where the evaporative member is spaced apart from the composition transfer surface.

ZZ. A dispenser comprising:
a. a housing;
b. a volatile liquid composition reservoir;
c. a fluid transfer member in fluid communication with the volatile liquid composition reservoir, and comprising a composition transfer surface, the fluid transfer member comprising a porous wicking element comprising sintered polymeric particles;
d. an evaporative member in contact with the composition transfer surface, the evaporative member comprising a woven metal mesh; and
e. a heating element affixed to the evaporative member for conductively transferring heat to the evaporative member and the composition transfer surface.

AAA. A dispenser comprising:
a. a housing;
b. a volatile liquid composition reservoir;
c. a fluid transfer member in fluid communication with the volatile liquid composition reservoir, and comprising a composition transfer surface;
d. an evaporative member in contact with the composition transfer surface, the evaporative member comprising a mesh; and
e. a heating element for transferring heat to the evaporative member,
wherein the volatile liquid composition reservoir, the fluid transfer member, and the evaporative member are removably contained within the housing and replaceable with another volatile liquid composition reservoir, fluid transfer member, and evaporative member; and
wherein the heating element is permanently contained within the housing.

BBB. A dispenser comprising:
a. a volatile liquid composition reservoir;
b. a fluid transfer member in fluid communication with the volatile liquid composition reservoir, and comprising a composition transfer surface capable of releasing some of a contained volatile liquid composition in a liquid state and/or vapor state;
c. an evaporative member movable between a first position in contact with the composition transfer surface and a second position separated from the composition transfer surface; and
d. a heating element,
wherein, when the evaporative member is in the second position, the heating element is in contact with the evaporative member and, when the heating element is activated, heat is conductively transferred to the evaporative member.

CCC. The dispenser as disclosed in AAA, wherein, when the evaporative member is in the first position, a portion of the contained volatile liquid composition in the liquid state moves from the fluid transfer member to the evaporative member until the evaporative member reaches a maximum load or until the evaporative member is moved out of contact with the fluid transfer member. DDD. The dispenser as disclosed in AAA or BBB, wherein, when the evaporative member is in the second position, no portion of the contained volatile liquid composition in the liquid state moves from the fluid transfer member to the evaporative member.

The dispensers herein are useful for delivering volatile compositions that include a perfume mixture comprising one or more perfume raw materials. Suitable perfume raw materials are disclosed in U.S. Pat. Nos. 5,663,134; 5,670,475; 5,783,544; 5,939,060; and 6,146,621. The volatile composition may include various different PRMs. Exemplary PRMs are listed in TABLE 1 below.

TABLE 1
Perfume Raw Materials

| CAS No. | Name | CAS No. | Name |
|---|---|---|---|
| 31375-17-4 | 1-(P-Menthen-6(2)-Yl)-1- | 93-28-7 | Eugenyl Acetate |
| | Propanone | | |
| 68991-97-9 | 1,2,3,4,5,6,7,8-Octahydro-8,8- | 1195-79-5 | Fenchone |
| | Dimethyl-2-Naphthaldehyde | | |
| 1192-88-7 | 1-Cyclohexene-1- | 137-03-1 | Fleuramone |
| | Carboxaldehyde | | |
| 66327-54-6 | 1-Methyl-4-(4-Methylpentyl)-3- | 71077-31-1 | Floral Super |
| | Cyclohexenecarbaldehyde | | |
| 95962-14-4 | 2-(2-(4-Methyl-3-Cyclohexen-1- | 67634-14-4 | Floralozone |
| | Yl)Propyl)-Cyclopentanone | | |
| 74338-72-0 | 2,4,4,7-Tetramethyl-Oct-6-En-3- | 125109-85- | Florhydral |
| | One | 5 | |
| 1335-66-6 | 2,4,6-Trimethyl-3-Cyclohexene- | | Formyl Tricyclodecan |
| | 1-Carboxaldehyde | | |
| 25152-84-5 | 2,4-Decadienal | 14765-30-1 | Freskomenthe |
| 68039-49-6 | 2,4-Dimethyl-3-Cyclohexen-1- | 6413-10-1- | Fructone |
| | Carbaldehyde | | |
| 68039-49-6 | 2,4-Dimethyl-3-Cyclohexene-1- | 68912-13-0 | Frutene |
| | Carboxaldehyde | | |
| 15764-16-6 | 2,4-Dimethylbenzaldehyde | 706-14-9 | Gamma Decalactone |
| 68737-61-1 | 2,4-Dimethylcyclohex-3-Ene-1- | 74568-05-1 | Gamma Undecalactone |
| | Carbaldehyde | | |
| 142-83-6 | 2,4-Hexadienal | 79-76-5 | Gamma-Ionone |
| 30361-28-5 | 2,4-Octadienal | 127-51-5 | Gamma-Methyl Ionone |
| 24048-13-3 | 2,6,10-Trimethyl-5,9- | 104-50-7 | Gamma-Octalactone |
| | Undecadien-1-Al | | |
| 141-13-9 | 2,6,10-Trimethyl-9-Undecenal | 108-29-2 | Gamma-Valero Lactone |
| 116-26-7 | 2,6,6-Trimethyl-1,3-Diene | 29214-60-6 | Gelsone |
| | Methanal | | |
| 472-66-2 | 2,6,6-Trimethyl-1-Cyclohexene- | 5392-40-5 | Geranial |
| | 1-Acetaldehyde | | |
| 106-72-9 | 2,6-Dimethyl-5-Heptenal | 57934-97-1 | Givescone |
| 26370-28-5 | 2,6-Nonadienal | 111-30-8 | Glutaraldehyde |
| 103-95-7 | 2.Methyl-3(P-Isopropylphenyl)- | 111-30-8 | Glutaric Aldehyde |
| | Propionaldehyde | | |
| 42370-07-0 | 2-Acetyl-3,3-Dimethyl- | 34902-57-3 | Habanolide |
| | Norbornane | | |
| 112-54-9 | 2-Dodecanal | 24851-98-7 | Hedione |
| 613-69-4 | 2-Ethoxybenzaldehyde | 1205-17-0 | Helional |
| 97-96-1 | 2-Ethylbutyraldehyde | 120-57-0 | Heliotropin |
| 6728-26-3 | 2-Hexenal | 141773-73- | Helvetolide |
| | | 1 | |
| 101-86-0 | 2-Hexyl 3-Phenyl Propenal | 111-71-7 | Heptanal |
| 90-02-8 | 2-Hydroxy Benzaldehyde | 79-78-7 | Hexalon |
| 35158-25-9 | 2-Isopropyl-5-Methyl-2-Hexenal | 66-25-1 | Hexenal |
| 101-39-3 | 2-Methyl 3-Phenyl Propenal | 101-86-0 | Hexyl Cinnamic Aldehyde |
| 96-17-3 | 2-Methyl Butyraldehyde | 2349-07-7 | Hexyl Iso-Butyrate |
| 19009-56-4 | 2-Methyl Deca-1-Al (2 Methyl | Specialty | Hs Raspberry |
| | Decanal) | | |
| 123-15-9 | 2-Methyl Valeraldehyde | 90-87-9 | Hydrotropaldehyde |
| 110-41-8 | 2-Methyl-1-Undecanal | 107-75-5 | Hydroxycitronellal |
| 623-36-9 | 2-Methyl-2-Pentenal | 120-72-9 | Indole |
| 1205-17-0 | 2-Methyl-3-(3,4- | 1337-83-3 | Intreleven Aldehyde |
| | Methylenedioxyphenyl)Propanal | | |
| 41496-43-9 | 2-Methyl-3- | 14901-07-6 | Ionone Beta |
| | Tolylproionaldehyde, 4- | | |
| | Dimethylbenzenepropanal (4- | | |
| | Dimethyl Benzenepropanal) | | |
| 80-54-6 | 2-Methyl-4-T- | 1335-66-6 | Iso Cyclocitral |
| | Butylphenyl)Propanal | | |
| 123-15-9 | 2-Methylpentanal | 1335-66-6 | Iso Cyclocitral |
| 623-36-9 | 2-Methylpentenal | 95-41-0 | Iso Jasmone |
| 122-40-7 | 2-Pentyl-3-Phenylpropenoic | 659-70-1 | Iso-Amyl Iso-Valerate |
| | Aldehyde | | |
| 4411-89-6 | 2-Phenyl 2-Butenal | 78-84-2 | Isobutyraldehyde |
| 93-53-8 | 2-Phenylproprionaldehyde | 54464-57-2 | Isocyclemone E |
| 125109-85- | 3-(3-Isopropyl-Phenyl)- | 1335-66-6 | Iso-Cyclo Citral |
| 5 | Butyraldehyde | | |
| 103-95-7 | 3-(P-Isopropylphenyl)- | 70266-48-7 | Iso-Damascone |
| | Propionaldehyde | | |
| 4433-36-7 | 3,4,5,6-Tetrahydropseudoionone | 54464-57-2 | Iso-E-Super |
| 139-85-5 | 3,4-Dihydroxybenzaldehyde | 58430-94-7 | Iso-Nonyl Acetate |
| 120-14-9 | 3,4-Dimethoxybenzaldehyde | 590-86-3 | Isovaleraldehyde |
| 120-57-0 | 3,4-Methylene Dioxy | 101-86-0 | Jasmonal H |
| | Benzaldehyde | | |
| 134-96-3 | 3,5-Dimethoxy 4- | 41496-43-9 | Jasmorange |
| | Hydroxybenzaldehyde | | |
| 106-23-0 | 3,7-Dimethyl 6-Octenal | 2111-75-3 | L-4(1-Methylethenyl)-1- |
| | | | Cyclohexene-1- |
| | | | Carboxaldehyde |
| 107-75-5 | 3,7-Dimethyl Octan-1-Al | 112-54-9 | Lauric Aldehyde |
| 106-24-1 | 3,7-Dimethyl-2,6-Octadien-1-Al | 491-35-0 | Lepidine |
| 7492-67-3 | 3,7-Dimethyl-6-Octenyl | 68039-49-6 | Ligustral |
| | Oxyacetaldehyde | | |
| 121-32-4 | 3-Ethoxy 4- | 62518-65-4 | Lilestralis 33 |
| | Hydroxybenzaldehyde | | |
| 590-86-3 | 3-Methyl Butyraldehyde | 80-54-6 | Lilial |
| 107-86-8 | 3-Methyl-2-Butenal | | Lime Aldehyde |
| 55066-49-4 | 3-Methyl-5-Phenyl Pentanal | 78-70-6 | Linalool |
| 16630-52-7 | 3-Methylthiobutanal | 115-95-7 | Linalyl Acetate |
| 16251-77-7 | 3-Phenyl Butanal | 3720-16-9 | Livescone |
| 36306-87-3 | 4-(1-Ethoxyvinyl)-3,3,5,5,- | 51414-25-6 | Lyral |
| | Tetramethyl-Cyclohexanone | | |
| 122-48-5 | 4-(4-Hydroxy-3- | 80-54-6 | Lysmeral |
| | Methoxyphenyl)-2-Butanone | | |
| 31906-04-4 | 4-(4-Hydroxy-4-Methyl Pentyl)- | 67845-30-1 | Maceal |
| | 3-Cyclohexene-1- | | |
| | Carboxaldehyde | | |
| 4927-36-0 | 4-Damascol | 20407-84-5 | Mandarinal |
| 10031-82-0 | 4-Ethoxybenzaldehyde | 20407-84-5 | Mandarine Aldehyde |
| 4748-78-1 | 4-Ethyl Benzaldehyde | 39255-32-8 | Manzanate |
| 122-03-2 | 4-Isopropyl Benzaldehyde | 62518-65-4 | Mefloral |
| 621-59-0 | 4-Methoxy 3-Hydroxy | 55066-49-4 | Mefranal |
| | Benzaldehyde | | |
| 5703-26-4 | 4-Methylphenylacetaldehyde | 68991-97-9 | Melafleur |
| 18127-01-0 | 4-T- | 106-72-9 | Melonal |
| | Butylbenzenepropionaldehyde | | |
| 80-54-6 | 4-Tert-Butyl-Alpha-Methyl- | 30772-79-3 | Melozone |
| | Hydrocinnamaldehyde | | |
| 32210-23-4 | 4-Tertiary Butyl Cyclohexyl | 89-80-5 | Menthone |
| | Acetate | | |
| 30168-23-1 | 4-Tricyclo5210-2,6decylidene- | 62439-41-2 | Methoxy Melonal |
| | 8butanal | | |
| 37609-25-9 | 5-Cyclohexadecenone | 1504-74-1 | Methoxycinnamaldehyde |
| | | | (Ortho) |
| 33704-61-9 | 6,7-Dihydro-1,1,2,3,3- | 24851-98-7 | Methy-Dihydrojasmonate |
| | Pentamethyl-4(5h)-Indanone | | |
| 34131-99-2 | 6-Isopropyldecahydro-2- | 93-08-3 | Methyl Beta Naphthyl |
| | Naphtone | | Ketone |
| 62439-41-2 | 6-Methoxy-2,6- | 32388-55-9 | Methyl Cedrylone Major |
| | Dimethylheptanal | | |
| 107-75-5 | 7-Hydroxy-3,7-Dimethyl Octan- | 103-26-4 | Methyl Cinnamate |
| | 1-Al | | |
| 123-69-3 | 8-Hexadecenolide | 68480-14-8 | Methyl Cyclocitrone |
| 84697-09-6 | Acalea | 24851-98-7 | Methyl Dihydro Jasmonate |
| 75-07-0 | Acetaldehyde | 93-16-3 | Methyl Isoeugenol |
| 98-86-2 | Acetophenone | 110-41-8 | Methyl Nonyl Acetaldehyde |
| 141-13-9 | Adoxal | 112-12-9 | Methyl Nonyl Ketone |
| 19009-56-4 | Aldehyde C-11 MOA | 19009-56-4 | Methyl Octyl Acetylaldehyde |
| 110-41-8 | Aldehyde C12 MNA | 93-92-5 | Methyl Phenyl Carbinyl |
| | | | Acetate |
| 123-68-2 | Allyl Caproate | 119-36-8 | Methyl Salicylate |
| 122-40-7 | Alpha-Amylcinnamic Aldehyde | 122-00-9 | Methyl-Acetophenone |
| 6753-98-6 | Alpha-Caryophyllene | 93-08-3 | Methyl-Beta-Naphthyl- |
| | | | Ketone |
| 43052-87-5 | Alpha-Damascone | 96-17-3 | Methylbutyraldehyde |
| 101-86-0 | Alpha-Hexylcinnamaldehyde | 32388-55-9 | Methyl-Cedrenyl-Ketone |
| 127-41-3 | Alpha-Ionone | 32388-55-9 | Methyl-Cedrylone |
| 101-39-3 | Alpha-Methyl Cinnamic | 101-39-3 | Methylcinnamaldehyde |
| | Aldehyde | | |
| 127-42-4 | Alpha-Methyl Ionone | 110-93-0 | Methyl-Heptenone |
| 101-39-3 | Alpha-Methylcinnamaldehyde | 67633-95-8 | Methyl-Lavender-Ketone |
| 103-95-7 | Alpha-Methyl-P-Isopropyl | 7492-67-3 | Muget Aldehyde 50 |
| | Phenyl Propyl Aldehyde | | |
| 101-86-0 | Alpha-N-Hexyl- | 541-91-3 | Muscone |
| | Cinnamaldehyde | | |
| 80-56-8 | Alpha-Pinene | 33704-61-9 | Musk Indanone |
| 628-63-7 | Amyl-Acetate | 21145-77-7 | Musk Plus |
| 122-40-7 | Amyl Cinnamic Aldehyde | 37677-14-8 | Myrac Aldehyde |
| 495-85-2 | Amylaldehyde | 564-94-3 | Myrtenal |
| 123-11-5 | Anisaldehyde | 127-43-5 | N-Beta-Methyl Ionone |
| | | | Isomer |
| 123-11-5 | Anisic Aldehyde | 173445-65- | Neo Hivernal |
| | | 3 | |
| 5462-06-6 | Anisylpropanal | 56973-85-4 | Neobutenone |
| 100-52-7 | Benzaldehyde | 106-26-3 | Neral |
| 104-53-0 | Benzenepropanal | 124-19-6 | Nonanal |
| 119-61-9 | Benzophenone | 18829-56-6 | Nonenal |
| 140-11-4 | Benzyl Acetate | 86803-90-9 | Octahydro-5-Methoxy-4,7- |
| | | | Methano-1H-Indene-2- |
| | | | Carboxaldehyde |
| 100-51-6 | Benzyl Alcohol | 124-13-0 | Octanal |
| 120-51-4 | Benzyl Benzoate | 2548-87-0 | Octenal |
| 118-58-1 | Benzyl Salicylate | 54082-68-7 | Onicidal (Muguet |
| | | | Undecadienal) |
| 2550-26-7 | Benzyl-Acetone | 8028-48-6 | Orange Oil Tarocco |
| Specialty | Berry Wescorps | 16587-71-6 | Orivone |
| 65885-41-8 | Beta Methyl Benzenepropanal | 59323-76-1 | Oxane |
| 432-25-7 | Beta-Cyclocitral | 80-54-6 | P. T. Bucinal |
| 35044-68-9 | Beta-Damascone | 5471-51-2 | Para Hydroxy Phenyl |
| | | | Butanone |
| 928-96-1 | Beta-Gamma Hexanol | 67634-14-4 | Para-Ethyl-Alpha, Alpha- |
| | | | Dimethyl |
| | | | Hydrocinnamaldehyde |
| 14901-07-6 | Beta-Ionone | 100-06-1 | Para-Methoxy-Acetophenone |
| 128-37-0 | BHT | 98-53-3 | Para-Tert-Butyl- |
| | | | Cyclohexanone |
| 18127-01-0 | Bourgeonal | 106-02-5 | Pentadecanolide |
| 75147-23-8 | Buccoxime | 110-62-3 | Pentanal |
| 123-72-8 | Butyraldehyde | 111-30-8 | Pentanedial |
| 76-22-2 | Camphor | 2111-75-3 | Perillaldehyde |
| 5462-06-6 | Canthoxal | 103-60-6 | Phenoxy Ethyl Iso-Butyrate |
| 99-49-0 | Carvone | 101-48-4 | Phenyl Acetaldehyde |
| | | | Dimethyl Acetal |
| 55418-52-5 | Cassione (Heliotropin Acetone) | 4411-89-6 | Phenyl Butenal |
| Specialty | Cassis Base | 60-12-8 | Phenyl Ethyl Alcohol |
| 139-85-5 | Catechaldehyde | 103-48-0 | Phenyl Ethyl Iso-Butyrate |
| 3720-16-9 | Celery Ketone | 14371-10-9 | Phenyl Propenal, 3-Phenyl-2- |
| | | | Propenal |
| 104-55-2 | Cinnamic Aldehyde | 122-97-4 | Phenyl Propyl Alcohol |
| 103-54-8 | Cinnamyl Acetate | 122-78-1 | Phenylacetaldehyde |
| 6728-31-0 | Cis Heptenal | 564-94-3 | Pin-2-Ene-1-Carbaldehyde |
| 488-10-8 | Cis-Jasmone | 33885-51-7 | Pino Acetaldehyde |
| 5392-40-5 | Citral | 41724-19-0 | Plicatone |
| 106-23-0 | Citronellal | 123-11-5 | P-Methoxybenzene Aldehyde |
| 107-75-5 | Citronellal Hydrate | 101-39-3 | P-Methyl-Alpha- |
| | | | Pentylcinnamaldehyde |
| 106-22-9 | Citronellol | 107898-54- | Polysantol |
| | | 4 | |
| 7492-67-3 | Citronellyl Oxyacetaldehyde | 52474-60-9 | Precyclemeone B |
| 120-14-9 | Corps 4322 (Vanillin Methyl | 1191-16-8 | Prenyl Acetate |
| | Ether) | | |
| Specialty | Corps Iris | 123-38-6 | Propanal |
| 91-64-5 | Coumarin | 123-38-6 | Propionaldehyde |
| 122-03-2 | Cuminaldehyde | 90105-92-3 | Prunella |
| 68039-49-6 | Cyclal C | 104-09-6 | P-Tolylacetaldehyde |
| 103-95-7 | Cyclamen Aldehyde | 78-98-8 | Pyruvaldehyde |
| 7775-00-0 | Cyclemax | 82461-14-1 | Rhubafuran |
| 68738-96-5 | Cyclemone A | 116-26-7 | Safranal |
| 91462-24-7 | Cyclic Ethylene Dodecanedioate | 90-02-8 | Salicylaldehyde |
| 31906-04-4 | Cyclohexenyl-Carboxaldehyde | 41496-43-9 | Satinaldehyde |
| 502-72-7 | Cyclopentadecanone | 86803-90-9 | Scentenal |
| 103-95-7 | Cyclosal | 104-09-6 | Syringaldehyde |
| 103-95-7 | Cymal | 21944-98-9 | Tangerinal |
| 43052-87-5 | Damarose Alpha | 1322-58-3 | Tetrameran |
| 23696-85-7 | Damascenone | 22471-55-2 | Thesaron |
| 35044-68-9 | Damascone Beta | 21145-77-7 | Tonalid |
| 112-31-2 | Decanal | 18829-55-5 | Trans Heptenal |
| 4819-67-4 | Delphone | 24680-50-0 | Trans-4- |
| | | | Methoxycinnamaldehyde |
| 57378-68-4 | Delta-Damascone | 30168-23-1 | Tricyclodecylidenebutanal |
| 18479-58-8 | Dihydro Myrcenol | 10486-19-8 | Tridecanal |
| 17283-81-7 | Dihydro-Beta-Ionone | 16251-77-7 | Trifernal |
| 5988-91-0 | Dihydrocitronellal | 68039-49-6 | Triplal |
| 1128-08-1 | Dihydrojasmone | 67801-65-4 | Triplal Extra |
| 85-91-6 | Dimethyl Anthranilate | 27939-60-2 | Trivertal |
| 151-05-3 | Dimethyl Benzyl Carbinyl | 11245-8 | Undec-10-En-1-Al (10- |
| | Acetate | | Undecenal) |
| 10094-34-5 | Dimethyl Benzyl Carbinyl | 104-67-6 | Undecalactone |
| | Butyrate | | |
| 2550-11-0 | Dimethyl-Octenone | 81782-77-6 | Undecavertol |
| 5989-27-5 | D-Limonene | 112-44-7 | Undecanal |
| 34590-94-8 | Dowanol DPM Isomer | 110-62-3 | Valeraldehyde |
| 55418-52-5 | Dulcinyl | 121-33-5 | Vanillin |
| 30168-23-1 | Duplical | 20665-85-4 | Vanillin Isobutyrate |
| 75-07-0 | Ethanal | 65443-14-3 | Veloutone |
| | Eth-Me-Ph Glycidate Isomer | 120-14-9 | Veratraldehyde |
| 39255-32-8 | Ethyl 2 Methyl Pentanoate | 1728-46-7 | Verdone |
| 4940-11-8 | Ethyl Maltol | 88-41-5 | Verdox |
| 35044-59-8 | Ethyl Safranate | 88-41-5 | Verdox Major |
| 121-32-4 | Ethyl Vanillin | 66327-54-6 | Vernaldehyde |
| 7452-79-1 | Ethyl-2-Methyl Butyrate | 32210-23-4 | Vertenex |
| 105-95-3 | Ethylene Brassylate | 68039-49-6 | Vertocitral |
| 470-82-6 | Eucalyptol | 1335-46-2 | Xandralia (Methyl) |
| 97-53-0 | Eugenol | 472-66-2 | B-Homocyclocitral |

The perfume mixture may comprise one or more perfume raw materials selected from the group consisting of: dihydro myrcenol; dimethyl benzyl carbinyl acetate; ethyl vanillin; florhydral; nonanal; undecanal; vanillin; beta gamma hexanol; decanal; citronellol; and combinations thereof. The perfume mixture may also include one or more perfume raw materials selected from the group consisting of: 3-(1,3-Benzodioxol-5-yl)-2-methylpropanal, canthoxal, vanillin, ethyl vanillin, citral, ligustral, cinnamic aldehydes, and combinations thereof.

The volatile composition may comprise from greater than 10 wt. %, alternatively greater than 15 wt. %, alternatively greater than 20 wt. %, alternatively greater than 30 wt. %, alternatively greater than 40 wt. %, alternatively greater than 50 wt. %, alternatively greater than 60 wt. %, alternatively greater than 70 wt. %, alternatively greater than 85 wt. %, of a perfume mixture, alternatively about 10 wt. % to about 90 wt. %, alternatively about 20 wt. % to about 90 wt. %, alternatively about 30 wt. % to about 90 wt. %, based on the total weight of the volatile composition.

The volatile composition may include one or more carriers. The carrier may be aqueous or non-aqueous. The carrier may be selected from the group consisting of a solvent and/or a diluent. The carrier may be present in the volatile composition at a level of up to and including 80 wt. %, alternatively up to and including 70 wt. %, alternatively up to and including 60 wt. %, alternatively up to and including 50 wt. %, alternatively up to and including 40 wt. %, alternatively up to and including 30 wt. %, alternatively up to and including 20 wt. %, alternatively up to and including 15 wt. %, alternatively up to and including 10 wt. %, by total weight of the volatile composition.

The carrier may include a solvent, diluent, or combinations thereof. The solvent or diluent may be a glycol selected from the group consisting of propylene glycol, dipropylene glycol, tripropylene glycol. The solvent or diluent may be selected from the group consisting of: dipropylene glycol methyl ether (“DPM”), tripropylene glycol methyl ether (“TPM”), 3-methoxy-3-methyl-1-butanol (“MMB”), volatile silicone oil, and dipropylene glycol esters of methyl, ethyl, propyl, butyl, ethylene glycol methyl ether, ethylene glycol ethyl ether, diethylene glycol methyl ether, diethylene glycol ethyl ether, isopropyl myristate, or any VOC under the tradename of Dowanol™ glycol ether, and combinations thereof. The carrier may include water.

The volatile composition may be substantially free of volatile organic compounds (“VOCs”), meaning it has no more than about 18%, alternatively no more than about 6%, alternatively no more than about 5%, alternatively no more than about 1%, alternatively no more than about 0.5%, by weight of the composition, of VOCs. The composition may be free of VOCs.

As used in this specification and the appended claims, the singular forms “a”, “an”, and “the” include plural references unless the content clearly dictates otherwise. Thus, for example, “a volatile material” may include more than one volatile material.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be understood by those skilled in the art that various other changes and modifications can be made without departing from the spirit and scope of the present disclosure. It is therefore intended to cover in the appended claims all such changes and modifications that are within the scope of the present disclosure.

What is claimed is:

1. A dispenser comprising:
a. a volatile liquid composition reservoir;
b. a fluid transfer member in fluid communication with the volatile liquid composition reservoir, and comprising a composition transfer surface capable of releasing some of a contained volatile liquid composition in a liquid state and/or vapor state;
c. an evaporative member in contact with the composition transfer surface in at least a first position; and
d. a heating element affixed to the evaporative member, wherein heat is conductively transferred to both the evaporative member and the composition transfer surface.

2. The dispenser of claim 1, wherein the evaporative member comprises a mesh, wherein the mesh comprises a metal wire mesh.

3. The dispenser of claim 2, wherein the mesh is a woven mesh.

4. The dispenser of claim 2, wherein the mesh comprises from about 40 to about 500 openings per inch as measured in a first direction and from about 500 to about 3,000 openings per inch as measured in a second direction orthogonal to the first direction.

5. The dispenser of claim 2, wherein the mesh comprises a weave pattern selected from the group comprising plain Dutch weave, reverse plain Dutch weave, Dutch twilled weave, and reverse Dutch twilled weave.

6. The dispenser of claim 2, further comprising two electrical leads connectable to a power source, wherein the two electrical leads are connected to the heating element and not the mesh.

7. The dispenser of claim 2, wherein the heating element has a higher electrical resistance than the mesh at a given electrical current.

8. The dispenser of claim 1, wherein the heating element comprises a ring-shaped or disk-shaped heating element comprising two electrical leads soldered thereto.

9. The dispenser of claim 1, further comprising a rechargeable battery.

10. The dispenser of claim 9, wherein the dispenser continuously or discontinuously delivers a volatilized composition for a period of at least 12 hours.

11. The dispenser of claim 1, wherein the dispenser delivers dipropylene methyl ether at a rate of more than 75 mg/h when the heating element receives a power input of less than 1 W.

12. The dispenser of claim 1, wherein the evaporative member reaches a temperature of 95° C. or higher within 1 minute of the heating element receiving power input.

13. The dispenser of claim 1, further comprising a fan that operates during which time power is delivered to the heating element.

14. The dispenser of claim 1, further comprising:
e. a second volatile liquid composition reservoir;
f. a second fluid transfer member in fluid communication with the second volatile liquid composition reservoir, and comprising a second composition transfer surface capable of releasing some of a contained volatile liquid composition in a liquid state and/or vapor state;
g. a second evaporative member in contact with the second composition transfer surface; and
h. a second heating element affixed to the second evaporative member, wherein heat is conductively transferred to both the second evaporative member and the second composition transfer surface; and
wherein the dispenser is connectable to a power source and wherein power can selectively be directed to the first heating element, the second heating element, or directed to both of the first heating element and the second heating element.

15. The dispenser of claim 14, wherein a selected power input value to the first and second heating element can be programmed and stored on the dispenser, wherein the stored value can be sent to the dispenser wirelessly.

16. The dispenser of claim 1, further comprising a volatile liquid composition contained in the volatile composition reservoir, and wherein some of the volatile liquid composition is released from the both the composition transfer surface and evaporative member in a vapor state.

17. The dispenser of claim 1, wherein the volatile liquid composition reservoir and fluid transfer member are removably contained within a housing and replaceable with another volatile liquid composition reservoir and fluid transfer member, and wherein the evaporative member and the heating element are permanently contained within the housing.

18. The dispenser of claim 1, wherein the heating element comprises a first footprint area, wherein the evaporative member comprises a second footprint area, and wherein a ratio of the first footprint area to second footprint area is from about 0.08 to about 1.

19. The dispenser of claim 1, wherein the fluid transfer member comprises a porous wicking element comprising sintered polymeric particles.

20. The dispenser of claim 1, wherein the evaporative member is movable from the at least a first position to a second position spaced apart from the composition transfer surface.
